(19) United States
(12) Patent Application Publication
(10) Pub. No.: US 2011/0004182 A1
(43) Pub. Date: Jan. 6, 2011

(54) FASTENER TAB AND RELATED METHOD

(75) Inventors: Michael D. Hilston, Painesville, OH (US); Donald V. Varanese, Mentor, OH (US)

Correspondence Address: Avery Dennison Corporation;Amanda Wittine, 8080 Norton Parkway, 22-D, Mentor, OH, 44060, US

(73) Assignee: AVERY DENNISON CORPORATION, Pasadena, CA (US)

(21) Appl. No.: 12/602,242

(22) PCT Filed: May 28, 2008
(86) PCT No.: PCT/US08/65012

§ 371 Date: Sep. 13, 2010

Related U.S. Application Data
(60) Provisional application No. 60/940,823, filed on May 30, 2007.
(60) Provisional application No. 60/943,855, filed on Jun. 14, 2007.

(51) Int. Cl. A61F 13/62; A44B 18/00; B32B 37/02; B32B 38/04; B29C 65/00

(52) U.S. Cl. 604/391; 24/306; 156/60; 156/250; 156/227; 156/192

(57) ABSTRACT

A fastener tab including a fastening-tape-segment, a hook-tape-segment, a release-tape-segment, and an antipeel-tape-segment, with the release-tape-segment and the antipeel-tape-segment being formed from a single tape. The antipeel-tape-segment is configured to prevent a susceptible leading edge of an anchoring zone of the release-tape-segment from being subjected to peel-initiating forces during deployment of the fastener tab. An adhesive area can be included to releasably bond a fastening deployment zone and the release anchoring zone together to minimize pre-deployment flagging.

### CROSS-REFERENCE TO RELATED APPLICATIONS

Priority is claimed to U.S. Provisional Patent Application No. 60/940,823, filed on May 30, 2007, entitled “Diaper Fastener Tab,” by Michael D. Hilston and Donald V. Varanese, and U.S. Provisional Patent Application No. 60/943,855, filed on Jun. 14, 2007, entitled “Diaper Fastener Tab,” by Michael D. Hilston and Donald V. Varanese. Both U.S. Provisional Patent Application No. 60/940,823 and U.S. Provisional Patent Application No. 60/943,855 are incorporated by reference herein in their entirety.

### FIELD OF THE INVENTION

A fastener tab and related method including a fastening-tape-segment, a release-tape-segment, and a hook-tape-segment.

### BACKGROUND OF THE INVENTION

A common fastener tab design includes a fastening-tape-segment, a release-tape-segment, and a hook-tape-segment. The fastening-tape-segment has an anchoring zone that anchors the fastener tab to a first surface of a rear diaper edge and a deployment zone, which is pivoted away from a second surface of the diaper edge to deploy the fastener tab. The hook-tape-segment is attached to the deployment zone of the fastening-tape-segment and engages with a front diaper portion when fitting the diaper on a wearer. The release-tape-segment includes an anchoring zone, which anchors the fastener tab to the second surface of the diaper's rear edge, and a hinge zone, which is attached to the fastening deployment zone for movement therewith during deployment. A fastener manufacturer typically supplies a roll of tape laminate (including a fastening tape, a hook tape, and a release tape) to a diaper manufacturer. The tape laminate is segmented in the machine direction into a plurality of fastener tabs, which are then installed onto diapers.

### SUMMARY OF THE INVENTION

A fastener tab is provided with a tape-segment that is strategically situated to prevent the peel-susceptible leading edge of the release-tape-segment from being directly subjected to deployment forces. This antipeel-tape-segment and the release-tape-segment are formed from a single tape and completely partitioned from each other prior to deployment of the fastener tab. This single-tape-formation accurately positions the antipeel-tape-segment relative to the release segment without any complicated registration equipment and/or alignment procedures. The fastener tab can be constructed from tapes with continuous adhesive layers whereby no special pattern coating is necessary. An adhesive area can be provided (and easily modified or eliminated) to releasably adhere the fastening-tape-segment (in its deployment zone) and the release-tape-segment (in its anchoring zone) to minimize pre-deployment flagging.

An exemplary embodiment is a fastener tab in a pre-deployment condition that is configured to be coupled to a diaper having a diaper edge with a first surface and a second surface. The fastener tab includes a fastening-tape segment, a hook-tape-segment, a release-tape-segment, and an antipeel-tape segment. The fastening-tape-segment includes a first anchoring zone that is configured to anchor the fastener tab to the first surface of the diaper edge, and a deployment zone that is configured to be pulled away from the second surface of the diaper edge and to deploy the fastener tab after a deployment force is applied to the fastener tab. The hook-tape-segment is attached to the deployment zone. The release-tape-segment includes a second anchoring zone having a leading edge, the second anchoring zone is configured to anchor the fastener tab to the second surface of the diaper edge, and a hinge zone that is attached to the deployment zone. The antipeel-tape-segment is attached to the deployment zone. The antipeel-tape-segment is positioned to prevent the leading edge of the second anchoring zone from being subjected to the deployment force. The release-tape-segment and the antipeel-tape-segment are formed from a single tape.

In other, more detailed features of the invention, the fastening-tape-segment has a first side, the hinge zone and the antipeel-tape-segment overlie the first side of the fastening-tape-segment, and the second anchoring zone overlies the hinge zone and the antipeel-tape-segment. Also, the fastener tab can further include at least one mask-segment that is attached to the deployment zone. In addition, the fastening-tape-segment can have a distal edge, and the fastener tab can further include a fingerlift that is attached to the deployment zone of the fastening-tape-segment adjacent its distal edge.

In other, more detailed features of the invention, the fastener tab further includes an antiflag adhesive area that is configured to releasably adhere the deployment zone to the release-tape-segment or the second surface of the diaper edge. Also, the fastening-tape-segment can include a substrate and an adhesive layer that is coupled to the substrate, and the hook-tape-segment, the release-tape-segment, and the antipeel-tape-segment can be attached to the adhesive layer. In addition, the antiflag adhesive area can be configured to releasably adhere the deployment zone and the second anchoring zone together.

In other, more detailed features of the invention, the adhesive layer is continuous across and along the substrate. Also, an area of the adhesive layer can form the antiflag adhesive area. In addition, the fastener tab can further include a mask-segment that covers otherwise uncovered areas of the adhesive layer of the fastening-tape-segment.

In other, more detailed features of the invention, the antiflag adhesive area is positioned between the hinge zone of the release-tape-segment and the antipeel-tape-segment. Also, the fastener tab can further include a mask-segment positioned between the hinge zone of the release-tape-segment and the antipeel-tape-segment. In addition, the distal edge of the hook-tape-segment can be substantially aligned with the distal edge of the fastening-tape-segment. Furthermore, the distal edge of the hook-tape-segment can be inset with respect to the distal edge of the fastening-tape-segment. Also, the fastener tab can further include a fingerlift that is attached to the deployment zone of the fastening-tape-segment between the hook-tape-segment and the distal edge of the fastening-tape-segment.

In other, more detailed features of the invention, the hook-adjacent edge of the antipeel-tape-segment and/or the leading edge of the release-tape-segment are positioned immediately adjacent to the proximal edge of the hook-tape-segment. Also, both the hook-adjacent edge of the antipeel-tape-segment and the leading edge of the anchoring zone of the release-tape-segment can be positioned immediately adjacent to the proximal edge of the hook-tape-segment. In addition, the fastener tab can further include a mask-segment positioned between the antipeel-tape-segment and the proximal edge of the hook-tape-segment.

In other, more detailed features of the invention, the fastening-tape-segment is inelastic or elastic. Also, the fastening-tape-segment can be a one-piece segment or a multi-piece segment. In addition, the hook-tape-segment can be a one-piece segment or a multi-piece segment.

An exemplary method is a method of making a fastener tab. The method including the following steps: providing a single tape having a cross direction, partitioning the single tape in the cross direction into a release tape and an antipeel tape, compiling a tape laminate including a fastening tape, a hook tape, the release tape, and the antipeel tape, the tape laminate having a machine direction, and segmenting the tape laminate in the machine direction into the fastener tab.

In other, more detailed features of the invention, the tape-compiling step includes attaching the hook tape to the fastening tape. Also, the tape-compiling step can include attaching the single tape to the fastening tape before the single-tape-partitioning step. In addition, the tape-compiling step can include compiling a masking strip that is segmented into a mask-segment during the laminate-segmenting step. Furthermore, the tape-compiling step can include compiling a fingerlift strip that is segmented into a fingerlift during the laminate-segmenting step.

In other, more detailed features of the invention, the single tape includes an intermediate zone, an end zone, and an opposite end zone; the single tape partitioning step includes partitioning the opposite end zone from the intermediate zone and the end zone; and the intermediate zone and the end zone form the release tape, and the opposite end zone forms the antipeel tape. Also, the single tape can include a designed weakness between the intermediate zone and the opposite end zone.

In other, more detailed features of the invention, the single-tape-partitioning step includes breaking the designed weakness. Also, the single tape can include a fold line between the intermediate region and the end zone, and the method can further include the step of folding the single tape at the fold line. In addition, the method can further include the step of winding the compiled tape laminate into a roll. Furthermore, the roll can be compatible with automatic dispensing and diaper installation equipment.

In other, more detailed features of the invention, the hook-tape-attaching step is performed after the single-tape-attaching step. Also, the partitioning and compiling steps can be performed at one location, and the segmenting step can be performed at a remote location. In addition, the compiling step can be performed at one location and the partitioning and segmenting steps can be performed at a remote location. Furthermore, the one location can be a fastener manufacturing facility and the remote location can be a diaper manufacturing facility.

In other, more detailed features of the invention, the method further includes the step of folding the single tape between the opposite end zone and the intermediate zone, with the folding step being performed before the partitioning step. Also, the single-tape-attaching step can be performed after the folding step. In addition, the method can further include the step of forming the design weakness in the single tape. Furthermore, the design weakness can be a perforation line. Also, the designed-weakness-forming step can be performed before the single-tape-attaching step. In addition, the designed-weakness-forming step can be performed before the folding step.

In other, more detailed features of the invention, the single-tape-attaching step is performed after the step of folding the single tape at the fold line between the intermediate region and the end zone. Also, the step of folding the single tape can include folding the tape at two fold lines between the intermediate region and the end zone substantially simultaneously.

In other, more detailed features of the invention, the tape-compiling step includes attaching the hook tape to the fastening tape. Also, the tape-compiling step can include attaching the single tape to the fastening tape before the single-tape-partitioning step. In addition, the hook-tape-attaching step can be performed after the single-tape-attaching step. Furthermore, the tape-compiling step can include compiling a masking strip that is segmented into a mask-segment during the laminate-segmenting step. Also, the tape-compiling step can include compiling a fingerlift strip that is segmented into a fingerlift during the laminate-segmenting step.

Another exemplary embodiment is a diaper including a backsheet, a topsheet that is coupled to the backsheet forming a diaper edge having a first surface and a second surface, a liquid absorbent core enclosed within a combination formed from the topsheet and the backsheet, and at least one fastener tab as previously discussed.

In other, more detailed features of the invention, the diaper further includes a rear portion that is configured to cover the wearer's behind, a front portion that is configured to cover the wearer's front, and a crotch portion therebetween. The at least one fastener tab is attached to edges of the rear portion. Also, the anchoring zone of the fastening-tape-segment can be anchored to the inner surface of the diaper edge, and the anchoring zone of the release-tape-segment can be anchored to the outer surface of the diaper edge.

In other, more detailed features of the invention, a method of deploying a fastener tab included in the diaper includes the step of pivoting the deployment zone of the fastening-tape-segment outward to a fully extended condition.

Other features of the invention should become apparent to those skilled in the art from the following description of the preferred embodiments taken in conjunction with the accompanying drawings, which illustrate, by way of example, the principles of the invention, the invention not being limited to any particular preferred embodiment(s) disclosed.

### BRIEF DESCRIPTION OF THE DRAWINGS

These and other features, aspects, and advantages of the present invention will become better understood with reference to the following description, appended claims, and accompanying drawings, where:

FIG. 1 is a perspective view of a diaper with a pair of fastener tabs, the left-hand fastening tab being shown in a deployed condition and the right-hand fastener tab being shown in an installed condition.

FIG. 2 is an exaggerated exploded side view of the left-hand fastener tab.

FIGS. 3-8 are exaggerated views of the left-hand fastener tab in a pre-installation condition, a partial installation condition, an installation condition, an early deployment condition, a later deployment condition, and a deployed condition, respectively.

FIGS. 9A-9F are schematic diagrams showing deployment forces and the reaction of tape-segments thereto.

FIGS. 10A-10T are exaggerated side views of modified versions of the fastener tab in a pre-installation condition.

FIG. 11 is a schematic view of a system for making the fastener tabs.

FIGS. 12A-12C, 13A-13C, 14A-14C, 15A-15C, 16A-16D, 17, 18, and FIGS. 19A-19D are exaggerated side views from the machine direction or the cross-direction, these views showing tape-perforating, tape-folding, tape-laminating, laminate-slitting, tape-partitioning steps, and the laminates formed thereby.

FIG. 20 is a perspective view of a roll of a tape laminate, which can be segmented in the machine direction into separate fastener tabs.

FIGS. 21-23 are exaggerated side views in the machine direction showing tape laminates formed by modified sequences of the tape-laminating, laminate-slitting, and/or tape-partitioning steps.

FIGS. 24-25 are exaggerated side views of a tape laminate prior to the tape-partitioning step.

Unless otherwise indicated, the illustrations in the above figures are not necessarily drawn to scale.

### DETAILED DESCRIPTION OF THE INVENTION

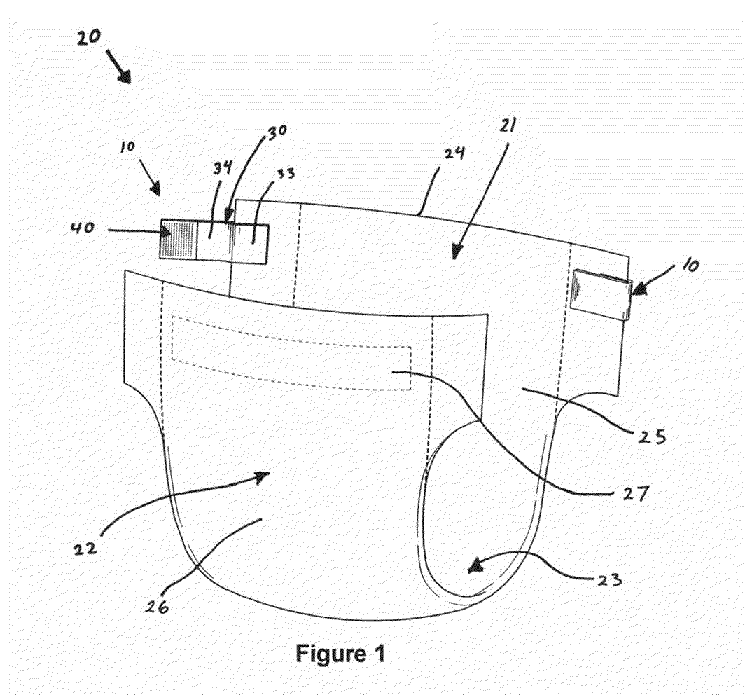

Referring now to the drawings, and initially to FIG. 1, a pair of fastener tabs 10 are shown installed on a diaper 20. The left-hand fastener tab 10 is shown in a deployed condition and the right-hand fastener tab 10 is shown in an installed condition. When the diaper 20 is packaged and sold by the diaper manufacturer, both fastener tabs 10 will be in the installed condition, and will be first placed in the deployment condition when the caretaker is fitting the diaper 20 on the wearer.

The illustrated diaper 20 comprises a liquid absorbent core enclosed within a liquid permeable backsheet and a liquid impermeable topsheet. The diaper 20 has a rear portion 21 which is intended to cover the wearer's behind, a front portion 22 which is intended to cover the wearer's front, and crotch portion 23 therebetween. These portions can be part of a one-piece or multi-piece chassis (e.g., the portions can comprise a chassis having side panels attached thereto to form the rear portion 21). In either or any event, the fastener tabs 10 are respectively attached to edges 24 of the rear portion 21. (As is best seen by referring briefly to FIGS. 4-6, the rear edges 24 each have a back surface 25 and a top surface 26). The fastener tabs 10 are arranged to provide closure about the wearer upon engagement with the diaper's front portion 22. The diaper 20 may include a landing strip 27 on its front portion 22 to facilitate this engagement or, in many cases, the fastener tabs 10 will directly engage the topsheet of diaper 20.

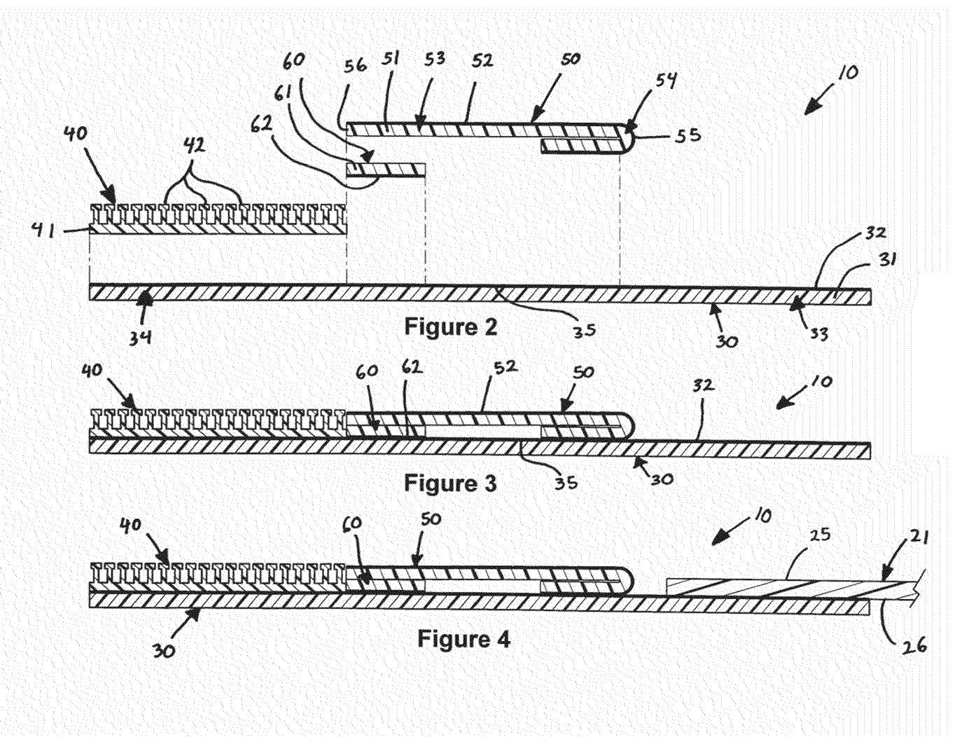

Referring now to FIG. 2, one of the fastener tabs 10 is shown in an exploded view that clearly illustrates its different components. Specifically, the fastener tab 10 includes a fastening-tape-segment 30, a hook-tape-segment 40, a release-tape-segment 50, and an antipeel-tape-segment 60. Although the fastener tabs 10 are shown in FIG. 1 as being generally rectangular in shape (whereby the segments/patch would usually also have a rectangular shape), such a regular and/or polygonal geometry is not necessary.

The fastening-tape-segment 30 includes a substrate 31 and an adhesive layer 32 on one side thereof, which together form an anchoring zone 33 and a deployment zone 34. The hook-tape-segment 40 includes a substrate 41 and hooks 42 carried thereby and projecting therefrom. The release-tape-segment 50 includes a substrate 51 and an adhesive layer 52 on one side thereof, which together form an anchoring zone 53 and a hinge zone 54. The antipeel-tape-segment 60 includes a substrate 61 and an adhesive layer 62 on one side thereof.

The thicknesses of the tape-segments 30/40/50/60 (and also segments 80 and 90 discussed below) are greatly exaggerated in the drawings. The thicknesses of these segments will usually be in the range of, for example, about 5 microns to about 100 microns. If these thicknesses were drawn to scale with the illustrated lengths, it would be more difficult to decipher and/or number the various segments, substrates, layers and/or zones of the fastener tab 10. Thus, the exaggerated thicknesses in the drawings are provided only for ease in explanation and to more clearly show the arrangement of the components relative to each other in the fastener tab 10. Also, with particular reference to assembled (rather than exploded) views of the fastener tab 10, while there may sometimes appear to be a slight space or gap between adjacent segments, zones and/or layers, they can be assumed to be in direct contact with each other unless otherwise noted.

Referring additionally to FIGS. 3-8, it can be seen that the hook-tape-segment 40, the hinge zone 54 of the release-tape-segment 50, and the antipeel-tape-segment 60 are attached to the deployment zone 34 of the fastening-tape-segment 30. The illustrated adhesive layers 32/52/62 are each continuous on the respective substrates 31/51/61, whereby the double adhesive layer 32/52 attaches the hinge zone 54 and the double adhesive layer 32/62 attaches the antipeel-tape-segment 60. If the hook-tape-segment 40 does not include its own adhesive layer (as shown), it will be attached by the single adhesive layer 32.

In a pre-installation condition (see FIG. 3), the fastening-tape-segment 30 is unfolded and linearly extends to define the fastener tab's length. The hook-tape-segment 40 occupies at least part of the distal region of the deployment zone 34 of the fastening-tape-segment 30. The release-tape-segment 50 and the antipeel-tape-segment 60 occupy at least part of the remaining region of the deployment zone 34. The anchoring zone 53 of the release-tape-segment 50, the hinge zone 54 of the release-tape-segment 50, and the antipeel-tape-segment 60 are positioned parallel with the fastening-tape-segment 30. The hinge zone 54 and the antipeel-tape-segment 60 overlie the fastening-tape-segment 30, and the anchoring zone 53 is folded over the hinge zone 54 and overlies it and the antipeel-tape-segment 60.

In the pre-installation condition, an adhesive area 35 is situated in the deployment zone 34 of the fastening-tape-segment 30 between the antipeel-tape-segment 60 and the hinge zone 54 of the release-tape-segment 50. While the schematic illustration shows a space between the adhesive area 35 and the anchoring zone 53, the aligned fastening/release areas are actually in direct contact and releasably adhered together. The substrate 51 of the release-tape-segment 50 is the contacting surface whereby this is a single-adhesive-layer bond, as opposed to the double-adhesive-layer bond 32/52 with the hinge zone 54 and the double-adhesive-layer bond 32/62 with the antipeel-tape-segment 60.

The fastener tab's elongated geometry and exposed-adhesive arrangement facilitate fastener production from a tape laminate (e.g., tape laminate 120 discussed below) and/or automatic installation of the fastener tab 10 onto the diaper 20. In the pre-installation condition of the illustrated fastener tab 10, only the adhesive 32/52 on the anchoring zones 33 and 53 are exposed. Also, the releasable bond formed by the adhesive area 35 with the anchoring zone 53 of the release-tape-segment 50 prevents flagging (i.e., pivoting outward relative to the hinge zone 54) of the pre-installation fastener tab 10.

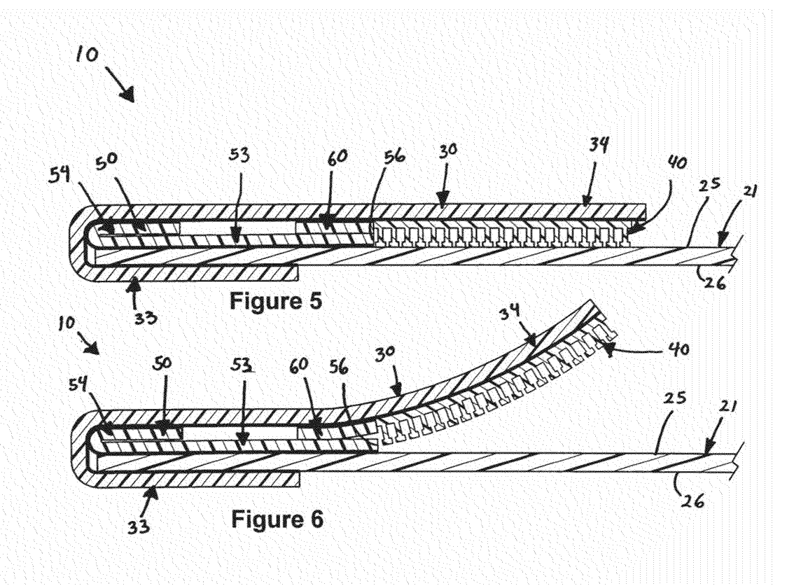

To install the fastener tab 10 onto the diaper 20, the anchoring zone 33 of the fastening-tape-segment 30 is anchored to one surface 26 of the diaper's rear edge 24 via the adhesive layer 32 (see FIG. 4). The deployment zone 34 of the fastening-tape-segment 30 (with the tape-segments 40/50/60 attached thereto) is then folded over the diaper's rear edge 24 and the anchoring zone 53 of the release-tape-segment 50 is anchored to the other surface 25 of the diaper rear's edge 24 via the adhesive 52 (see FIG. 5). The fastener tab 10 is thus anchored to both surfaces 25/26 of the diaper's rear edge 24 via the anchoring zones 33/53 forming what is often called a Y-bond. In the installed condition, the antiflag adhesive area 35 holds the deployment zone 34 of the fastening-tape-segment 30 against the anchoring zone 53 of the release-tape-segment 50 to prevent premature release or deployment.

To deploy the fastener tab 10, the deployment zone 34 of the fastening-tape-segment 30 is pivoted outward from the diaper back surface 25 to an extended fully deployed condition (see FIGS. 6-8). The releasable bond formed by the antiflag adhesive area 35 is broken during deployment and the zones 53/54 of the release-tape-segment 50 are unfolded. The anchoring zone 53 of the release-tape-segment 50 remains anchored to the back surface 25. The hinge zone 54 and the antipeel-tape-segment 60 remain attached to the fastening-tape-segment's deployment zone 34, with the anchor-hinge fold 55 opening to allow deployment.

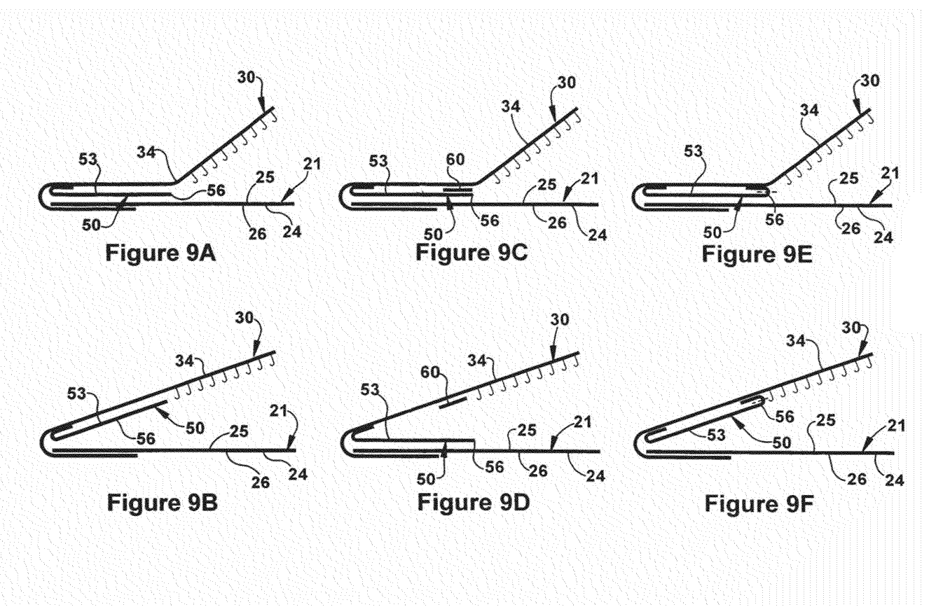

The role played by the antipeel-tape-segment 60 is best explained by referring to the deployment forces shown schematically FIGS. 9A-9F and remembering that, in most cases, the shear strength (parallel to the adhering surfaces) of an adhesive is much greater than its bond strength (perpendicular to the adhering surfaces). In FIGS. 9A-9B, the deployment forces are shown without an antipeel-tape-segment. In such a situation, the deployment zone 34 of the fastening-tape-segment 30 would be directly adhered to the leading edge 56 of the release-tape-segment 50. When the deployment pivot reaches this leading edge 56, the perpendicular component of the deployment force need only overcome the bond strength of the release-tape-segment 50 to the diaper surface 25 to cause havoc (see FIG. 9A). Specifically, the anchoring zone 53 of the release-tape-segment 50 will be lifted with the deployment zone 34 of the fastening-tape-segment 30 thereby destroying one side of the Y-bond to the diaper edge 24, either by separation of the release-tape-segment 50 from the diaper surface 25 or by tearing of the diaper portion 21 itself. Moreover, once the peel of the release-tape-segment 50 is initiated, shear forces will adhere the leading edge 56 of the release-tape-segment 50 to the fastening-tape-segment 30 (see FIG. 9B). The peel of the entire release-tape-segment 50 from the diaper surface 25 is almost inevitable.

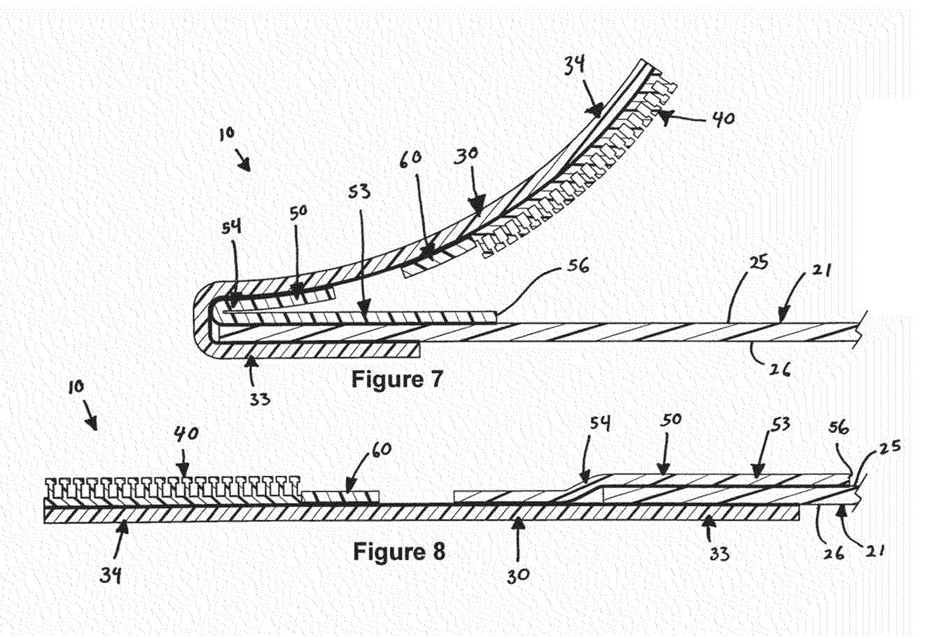

With the antipeel-tape-segment 60, a different scenario is presented as is shown schematically in FIGS. 9C-9D. With the tape-segment 60, the leading edge 56 of the release-tape-segment 50 is not directly subjected to deployment forces (see FIG. 9C, see also FIG. 6). The deployment zone 34 of the fastening-tape-segment 30 is directly adhered to the antipeel-tape-segment 60, not the leading edge 56 of the release-tape-segment 50. Since there is no adhesive bond between the release-tape-segment 50 and the antipeel-tape-segment 60, the antipeel-tape-segment 60 lifts easily away from the release-tape-segment 50 during deployment (see FIG. 9C). When the deployment pivot reaches the leading edge of the adhesive area 36 (see FIG. 9D, see also FIG. 7), the perpendicular component of the deployment force need only overcome the bond strength of the release-tape-segment 50 to the fastening-tape-segment 30. The parallel forces adhering the release-tape-segment 50 to diaper surface 25 minimize (if not eliminate) the risk of the anchoring zone 53 of the release-tape-segment 50 being lifted with the fastening-tape-segment 30.

As is explained in more detail below, the release-tape-segment 50 and the antipeel-tape-segment 60 are formed by partitioning a single tape (tape 170, discussed below) into two tapes (tapes 150 and 160 discussed below). In order for the tape-segment 60 to perform its peel preventing function, this partitioning must be complete prior to fastener deployment. Anything less than a partition, such as a designed weakness (e.g. a perforation line), does not decrease the peel susceptibility of the leading edge 56 of the anchoring tape-segment 53.

As is shown schematically in FIG. 9E-9F, with a designed-weakness-design, the deployment zone 34 of the fastening-tape-segment 30 is still adhered to the leading edge 56 of the release-tape-segment 50 at the time of deployment. When the deployment pivot reaches this leading edge 56, a resistance is encountered and a caretaker may even be inclined to give the fastener tab a “tug”. If this “tug” or deployment force is less than that required to break the designed weakness, but greater than that required to initiate peeling of the leading edge 56 of the release-tape-segment 50, the situation can go from bad to worse (see FIG. 9F). For this reason, some diaper manufacturers have a prejudice against such perforated fastener designs, be it justified or not.

As was explained above, the antiflag adhesive area 35 serves to hold the deployment zone 34 of the fastening-tape-segment 30 and the anchoring zone 53 of the release-tape-segment 50 in appropriate positions prior to deployment. Thus, the dimensions of the adhesive area 35 must be selected to fulfill this function. On the other hand, the bond provided by the adhesive area 35 must not be so great as to hinder separation of the zones 34 and 53 during deployment. It may also be undesirable to have “too much” post-deployment adhesive on the fastening-tape-segment as it can stick to the caretaker's fingers during fitting of the diaper 20.

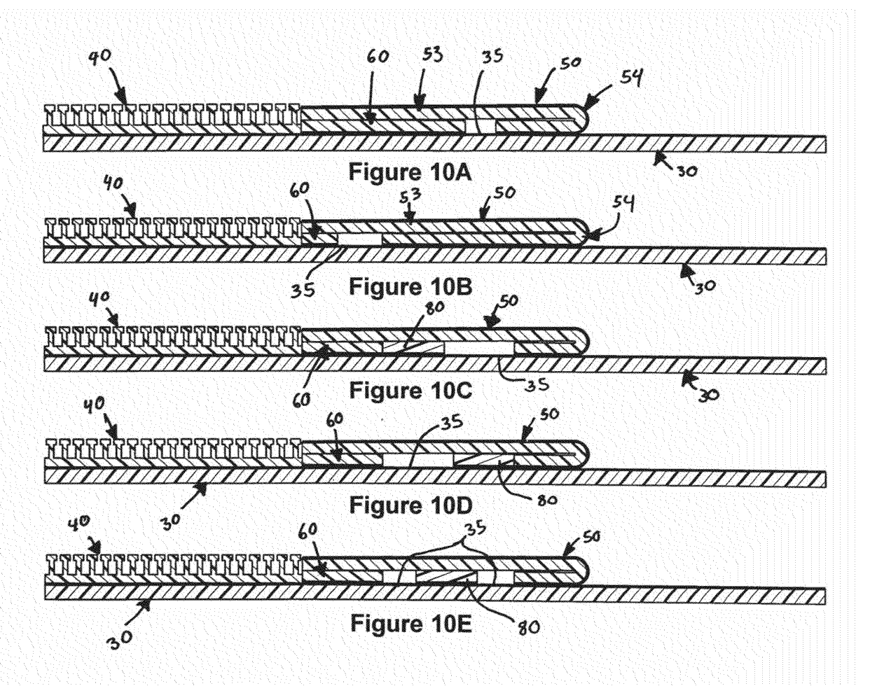

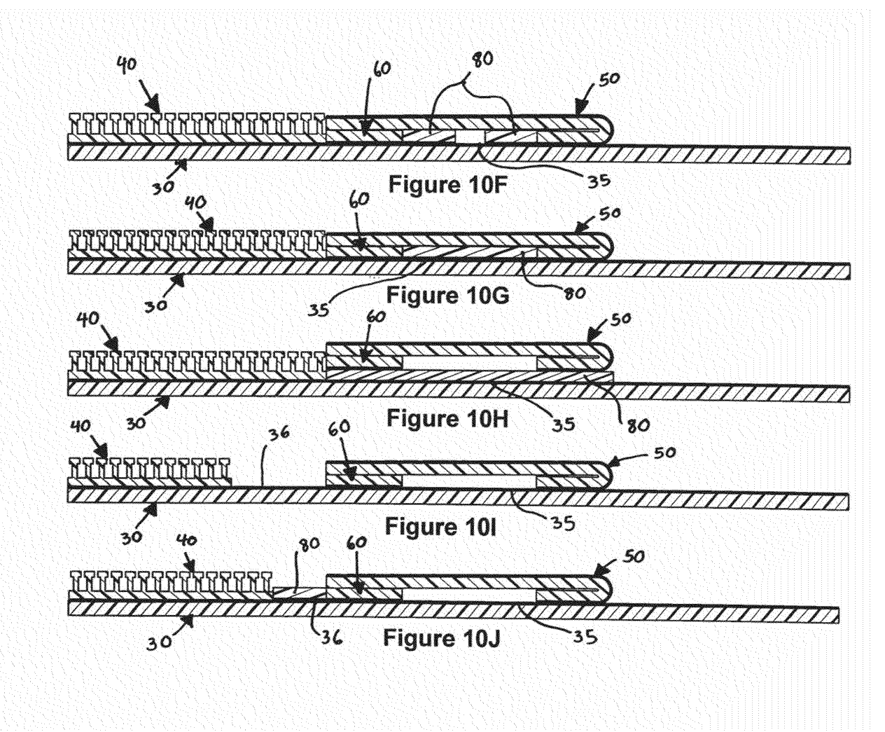

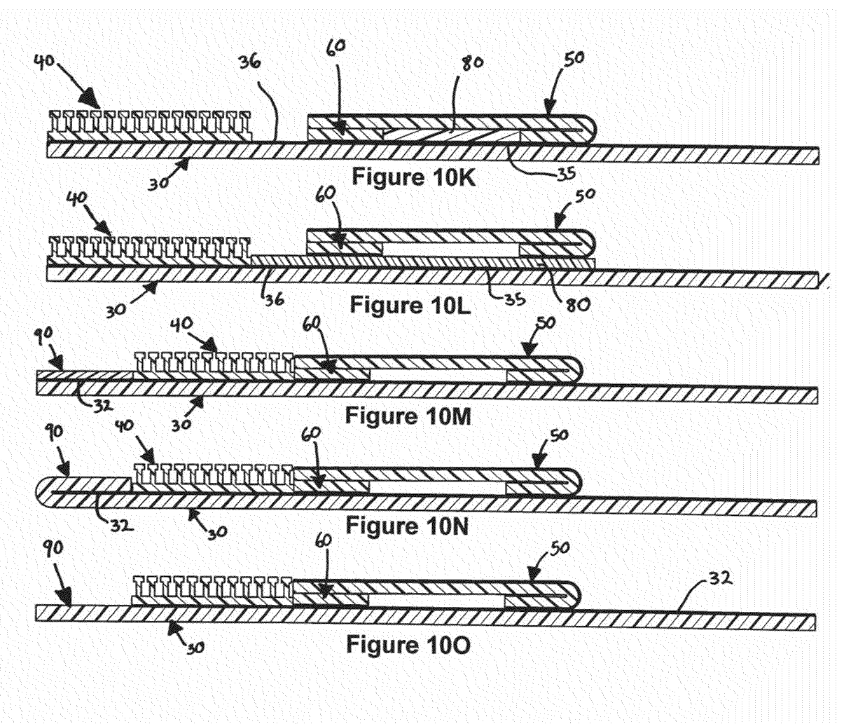

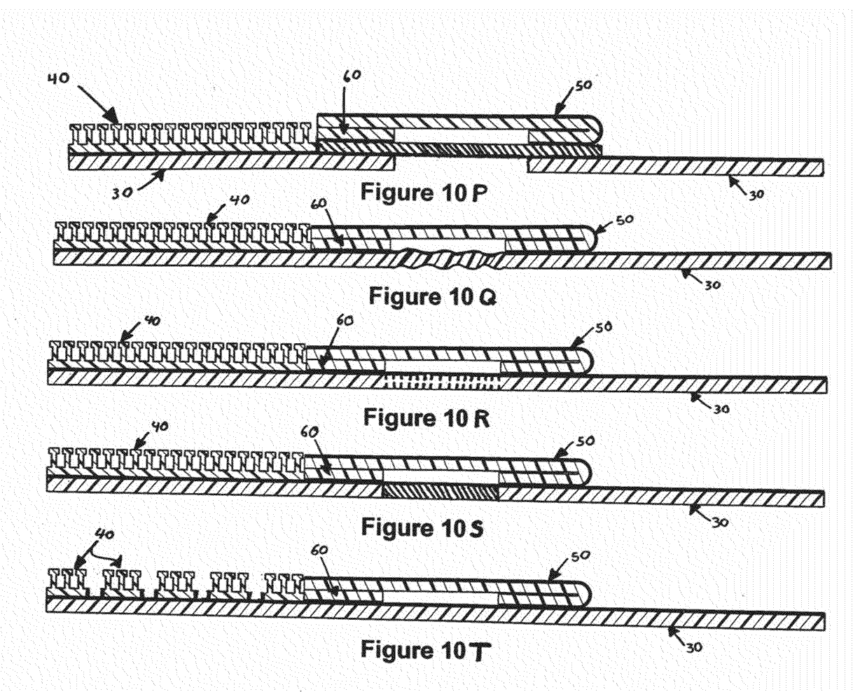

To change the geography (e.g., coverage, location) of the antiflag adhesive area 35, the size of the hinge zone 54 and/or the size of the antipeel-tape-segment 60 can be changed (see FIGS. 10A-10B). The release-tape-segment 50 and the antipeel-tape-segment 60 are formed by partitioning a single tape (tape 170, discussed below) into two tapes (tapes 150 and 160 discussed below). Thus, even with the same size tape, many different antiflag adhesive arrangements are possible. Additionally or alternatively, a mask-segment 80 can be used to reduce the size of the antiflag adhesive area 35 (see FIGS. 10C-10F). In fact, with some fastener tab designs, an antiflag adhesive area 35 may not be necessary and a mask-segment 80 can cover all exposed adhesive on the fastening-tape-segment 30 (see FIGS. 10G-10H). The latter would be true when, for example, the attachment of the hooks 42 to the diaper surface 25 is sufficient to prevent flagging.

In the fastener tab 10 shown in FIGS. 2-8, the antipeel-tape-segment 60 is positioned immediately adjacent the proximal edge of the hook-tape-segment 40. However, the anchoring zone 53 of the release-tape-segment 50 and/or the antipeel-tape-segment 60 can be spaced from the hook-tape-segment 40 (see FIGS. 10I-10L). This spacing could form the only or another antiflag adhesive area 36, which releasably adheres the fastening-tape-segment 30 to the diaper surface 25 (see FIGS. 10I and 10K). If such adherence is not desired, the space can instead be occupied by a mask-segment 80 (see FIGS. 10J and 10L).

In the embodiment illustrated in FIGS. 2-8, the distal edge of the hook-tape-segment 40 is aligned with the distal edge of the fastening-tape-segment 30. However, instead of the hook-tape-segment 40 extending to the distal end of the fastening-tape-segment 30, a fingerlift 90 can be employed (see FIGS. 10M-10O). The fingerlift 90 can be formed by a separate segment (see FIG. 10M), a fold-over of the distal end the fastening-tape-segment 30 (see FIG. 10N), or an adhesive-less area on the distal end of the fastening-tape-segment 30 (see FIG. 10O). Any of these fingerlifts (or other suitable fingerlifts) can be incorporated into any of the drawn, described, possible, and/or contemplated fastener tabs 10.

The fastening-tape-segment 30 need not be of a single-piece construction, and can instead include a plurality of subsegments joined together (e.g., an intermediate piece positioned between two end pieces) (see FIG. 10P). Such a multi-piece construction could be useful when the fastening-tape-segment 30 is intended to be an elastic component of the fastener 10. Specifically, for example, the intermediate piece could be elastic with the end pieces being inelastic. That being said, the fastening-tape-segment 30 need not have a multi-piece construction to be elastic. An entire one-piece segment 30 could be elastic. Alternatively, different districts of a one-piece fastening-tape-segment 30 could be elastic/inelastic by virtue of selective activation and/or composition (see FIGS. 10Q-10S).

The hook-tape-segment 40 also can have a multi-piece construction if, for example, hooks are to be positioned in lanes or rows (see FIG. 10T). But the release-tape-segment 50 and the antipeel-tape-segment 60 likely will have a one-piece construction because of the way they are formed (i.e., from a single tape 170). Multi-piece segments can be incorporated into any of the drawn, described, possible, and/or contemplated fastener tabs 10.

The location, size, or strength of adhesive areas on the fastening-tape-segment 30 and/or the release-tape-segment 50 could instead be accomplished by pattern-coating of an adhesive, or a plurality of adhesives with different strengths, on the substrate 31/51. Such pattern-coating, however, is often not preferred by fastener manufacturers, as it requires special equipment, complicated registration procedures, and other production headaches. Thus, one advantage of the fastener tab 10 is that it can be constructed of tape-segments 30/50/60 with continuous adhesive layers 32/52/62. “Continuous” in the present context refers to the fact that adhesive is not specifically applied to certain areas or zones of a substrate tape-segment/tape, but not others, according to the function of the area/zone in the fastener tab 10. Thus, a continuous layer can be, for example, an uninterrupted coating of adhesive or a regular/random coating pattern wherein the pattern is not dependent upon its application zone.

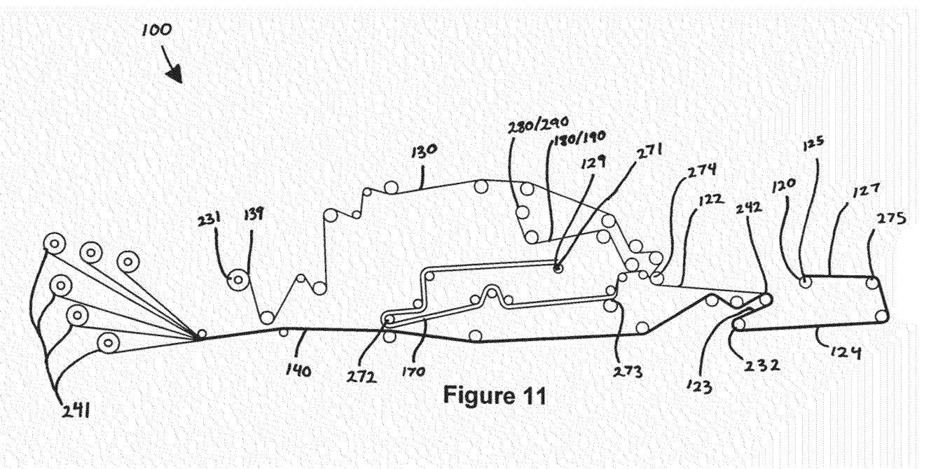

Referring now to FIG. 11, a system 100 is shown wherein a fastening tape 130, a hook tape 140, a release tape 150, and an antipeel tape 160 are compiled together to form a final tape laminate 110, which can be segmented into a plurality of the fastener tabs 10. The fastening tape 130 forms the fastening-tape-segments 30, the hook tape 140 forms the hook-tape-segments 40, the release tape 150 forms the release-tape-segments 50, and the antipeel tape 160 forms the antipeel-tape-segments 60 in the completed fastener tabs 10. The release tape 150 and the antipeel tape 160 are formed from a single tape 170, which is slit (in the machine direction) into the release and antipeel tapes 150 and 160, respectively. As was indicated above, the fastening-tape-segments 30/50/60 can have continuous adhesive layers 32/52/62 whereby the tapes 130 and 170 can have continuous adhesive layers 132/172.

In the illustrated embodiment, six tape laminates 110 are simultaneously made. The six fastening tapes 130 initially include a single webstock 139 (having a width representing six times the length of the fastening-tape-segment 30 or the width of the tape 130) dispensed from a single supply roll 231. Six hook tapes 140 are supplied from six supply rolls 241, and the six tapes 170 are supplied from six supply rolls 271. The fastening tapes 130 could instead be supplied from a plurality of webs (corresponding to the number of tape laminates 120 or a multiple thereof), the hook tapes 140 could be supplied in single or plural webs slit downstream, and/or the tapes 170 could be supplied in single or plural webs slit downstream.

The fastening tape 130 (and thus the webstock 139) includes a substrate 131 with an adhesive layer 132 thereon. The hook tape 140 includes a substrate 141 and hooks 142 carried by and projecting therefrom. The tape 170 includes a substrate 171 and an adhesive layer 172. In the illustrated orientation or the system 100, the adhesive 132 is a lower layer of the tape 130, the hooks 142 project downward in the hook tape 140, and the adhesive 172 is the upper layer of the tape 170.

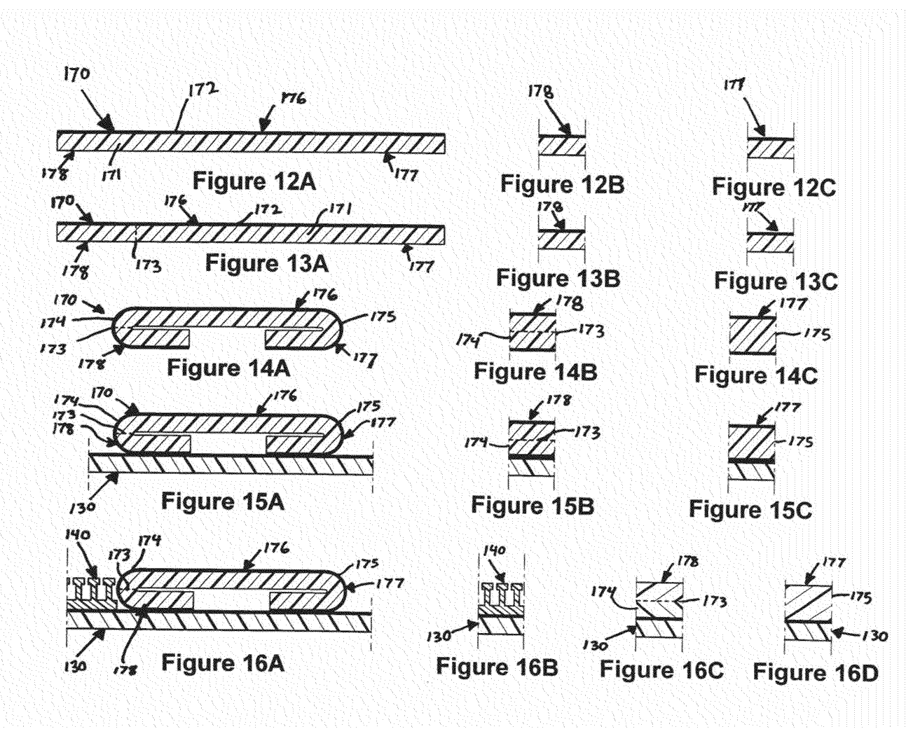

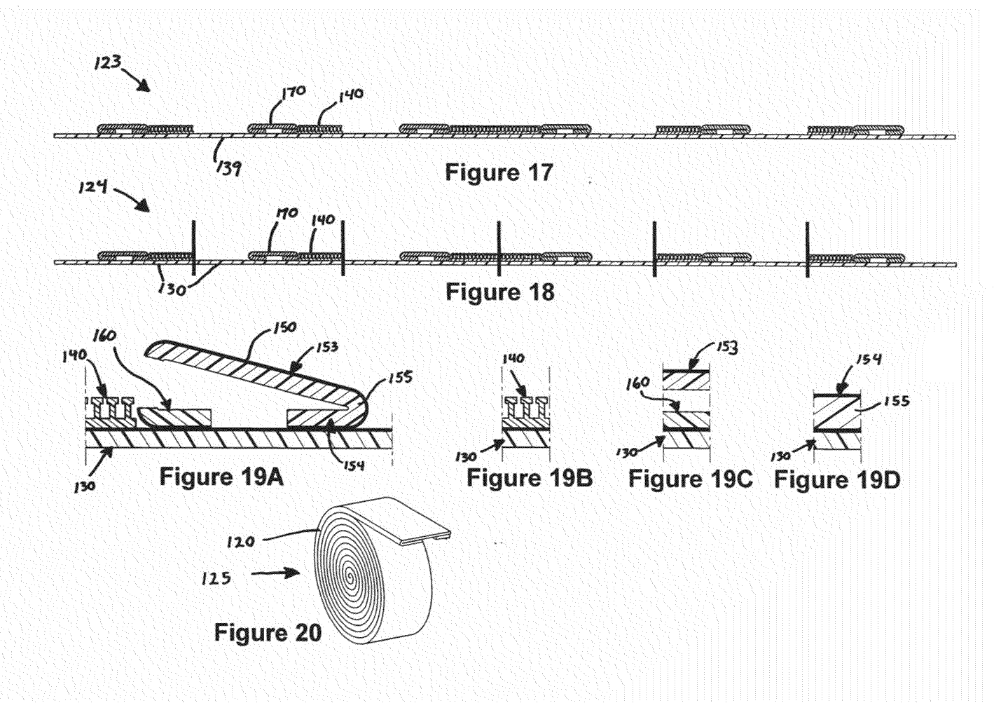

The single tape 170 passes from its supply roll 271 to a station 272 whereat it is provided with a designed weakness 173 (e.g., perforations) at a location corresponding to the partition between the release tape 150 and the antipeel tape 160 (see FIGS. 12A-12C and FIG. 13A-13C). The single tape 170 then passes to a station 273 whereat it is folded at a fold line 174 (at the same location as the designed weakness 173) and a fold line 175 (see FIGS. 14A-14C). The fold lines 174/175 can be formed at the same time/station (as shown) or they can be formed sequentially and/or at different stations.

The perforated fold line 174 is positioned between a zone 176 and a zone 177 of the tape 170 and the fold line 175 is positioned between the zone 176 and the zone 178 of the tape 170. The zone 176 corresponds to the anchoring zone 53 of the release-tape-segment 50, the zone 177 corresponds to the antipeel-tape-segment 60, and the zone 178 corresponds to the hinge zone 54 of the release-tape-segment 50. In the illustrated orientation, the substrate 171 will form the lower layer and the adhesive 172 will form the upper layer of the zone 176. The substrate 171 will form the upper layer and the adhesive 172 will form the lower layer in each of the zones 177 and 178.

The folded tape 170 is laminated to the webstock 139 at a laminating station 274 to form six lanes of a two-tape intermediate laminate 122 (see FIG. 11 and FIGS. 15A-15C). The hook tape 140 is laminated to the webstock 139 at a laminating station 242 to form six lanes of a three-tape intermediate laminate 123 (see FIG. 11 and FIGS. 16A-16D). In the illustrated laminate 123, the tapes 140/170 are symmetric relative to the centerline of the webstock 139 with the center two hook tapes 140 abutting against each other. However, other tape arrangements are certainly possible and contemplated. If the fastener tabs 10 are to include a mask-segment 80 and/or fingerlift 90, corresponding strips 180/190 can be provided and laminated at stations 274/242. The intermediate laminate 123 is slit into six lanes at a slitting station 232 to form the six fastening tapes 130 and six three-tape laminates 124 (see FIG. 18).

The six laminates 124 are then each passed through a partitioning station 275 whereat the folded tape 170 is partitioned into the release tape 150 and the antipeel tape 160 (see FIG. 11 and FIGS. 19A-19D). This partitioning can be accomplished by breaking the designed weakness 173 by applying a tearing pressure thereto by a partitioning device 275. A single partitioning device 275 may be provided for each tape laminate 124, or adjacent pairs of tape laminates 124 can share double-edged partitioning device 275. In any event, this completes the production of the final tape laminate 120 and its cross-section mirrors that of the fastening tab 10 in its pre-installation condition (see FIG. 3). The final tape laminate 120 can then be wound into a roll 125 for shipment to a diaper manufacturer whereat it can be segmented in the machine direction into individual fastener tabs 10 for installation on diapers 20.

One immediately apparent advantage of forming the release tape 150 and the antipeel tape 160 from the single tape 170 is that it reduces the number of starting materials, a welcome reduction from most fastener manufacturers' perspective. Also, as was indicated above, many different antiflag adhesive arrangements are possible, even with the same size tape. However, this single-tape formation affords some other, and perhaps more significant, benefits. The single tape 170 (as opposed to two separate tapes 150 and 160), especially in its folded form, provides processing stability during pre-hook-lamination stages. Also, the single-tape structure of what is to become the two tapes 150/160 prevents wandering of these tapes relative to each other and/or the fastening tape 130. Additionally, a more accurate placement of the hook tapes is often possible as the fold 174 forms a positive boundary for such placement.

Furthermore, the single-tape-formation of the release tape 150 and the antipeel tape 160 essentially guarantees that the antipeel-tape-segment 60 will be properly positioned in the fastener tape 10. More specifically, the folding and partitioning steps insure that the antipeel-tape-segment 60 will be situated in alignment with the leading edge 56 of the release-tape-segment 50 to perform its peel-preventing function. Significantly, this positioning is accomplished without any complicated registration equipment and/or alignment procedures.

In the above-described process, the tape 170 is provided with a designed weakness 173, which is broken to partition the tape 170 into tapes 150 and 160. However, the step of providing the tape 170 with a designed weakness is not necessary to achieve the advantages of the single-tape formation of tapes 150 and 160. The partitioning step could be performed, for example, by severing an intact tape 170 into the tapes 150 and 160 with a blade or other cutting device. The same partitioning device 275 (perhaps with a sharper blade) could be used to perform this severing step.

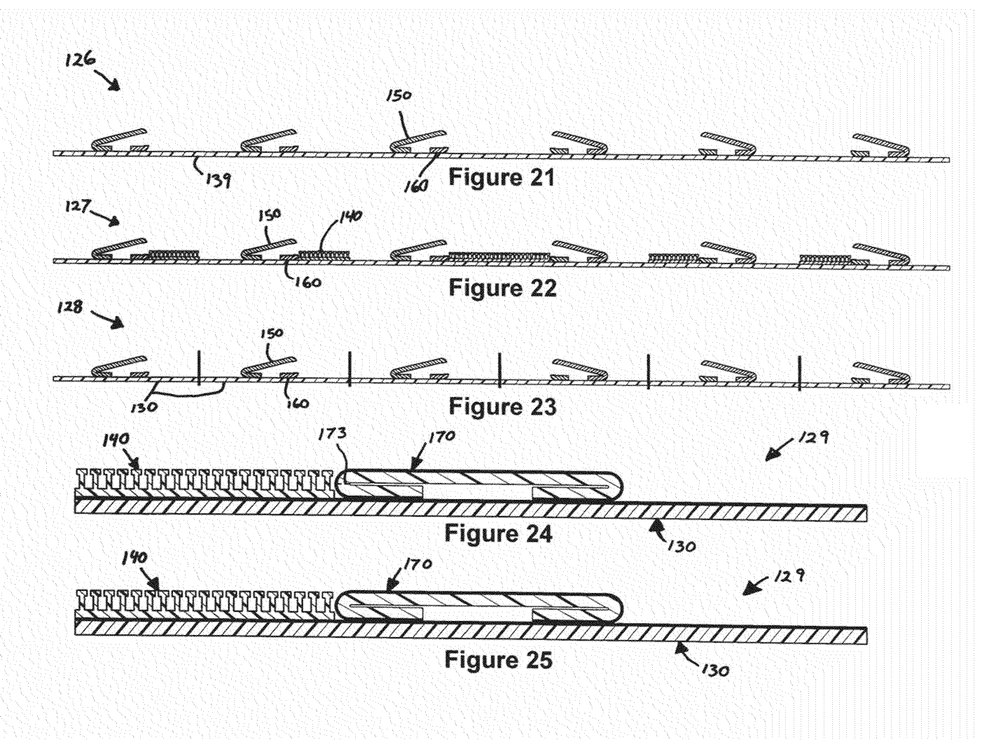

The tape lamination steps, the web-slitting step, and/or the tape-partitioning step can be performed sequentially in any suitable order and some steps may be able to be performed simultaneously, and/or at the same station. For example, the tape-partitioning step could occur prior to the hook lamination step and prior to the web-slitting step (see FIG. 21) to form an intermediate laminate 126, after the hook-lamination step but before the web-slitting step (see FIG. 22) to form an intermediate laminate 127, or after the web-slitting step but before the hook-lamination step to form an intermediate laminate 128 (see FIG. 23). In each case, the tape-partitioning step occurs prior to the tape laminate 120 being wound onto the dispensing rolls 125. Thus, the separation of the release tape 150 and the antipeel tape 160 is complete prior to shipment of the tape laminate 120 to the diaper manufacturer, and thus prior to fastener tab installation and prior to fastener tab deployment.

The important feature of the fastener tab 10 is the complete partitioning of the release tape 150 and the antipeel tape 160 prior to deployment of the fastener tab. As such, the tape-partitioning step could be performed by the diaper manufacturer, rather than the fastener manufacturer. In the latter case, the fastener manufacturer could provide a tape laminate 129 with a perforated tape 170 (see FIG. 24) or a tape laminate 129 with the tape 170 intact (see FIG. 25). The diaper manufacturer would then partition the tape 170 into the tapes 150/160 prior to installation of fastener tabs 10 onto diapers 20, as such a partitioning step may be convenient to incorporate into dispensing, fastener-segmenting, or other in-line installation steps. It may also be possible in certain production settings for the diaper manufacturer to partition the tape 170 into the tapes 150 and 160 (or segments 50 and 60) after installation of the fastener tabs 10 onto diapers 20.

The substrates 31/131, 51/151, 61/161, 171 can be made of cloth, kraft paper, cellophane film, non-woven webs, polymeric films (e.g., polypropylene, polyvinyl chloride, polyethylene terephthalate, and polyethylene) or other suitable materials or laminates. The non-adhesive sides of the substrates can include release coatings (e.g., a silicone coating, a carbamate coating, etc.) if such is necessary to prevent blocking issues during assembly, storing, and/or dispensing the fastener tabs 10 for installation on the diaper 20. If the substrates 31/131 are to be elastic, substrate selection could include extruded or coextruded elastic films that are monolayers or that include suitable skins, backings, or release linings.

The adhesive layers 32/132, 52/152, 62/162, 172 can be any conventional adhesive, including pressure sensitive adhesives and non-pressure sensitive adhesives. Suitable pressure sensitive adhesives include acrylic resin and natural or synthetic based rubber adhesives. As was indicated above, the fastener tab 10 can be advantageously constructed of tape-segments 30/50/60 with continuous adhesive layers 32/52/62, and thus formed from tapes 130/150/160/170 with continuous adhesive layers 132/152/162/172. That being said, patterned-coated adhesive layers are certainly possible and contemplated, and may even be preferred in certain situations.

The hook substrate 41/141 can be made of any material that is compatible with the hook-production process and accommodates its hook-carrying function. The hooks 42/142 can be made of a plastic, metal or other material and formed by, for example, molding or stamping. If the substrate 41/141 and the hooks 42/142 are separately formed, the hook substrate 41/141 can be made of, for example, cloth, kraft paper, cellophane film, non-woven webs, and/or polymeric films. Attachment of the hooks 42/142 to a separately-formed substrate 41/141 can be accomplished, for example, by adhesive bonding or hook-embedment. Alternatively, the substrate 41/141 and the hooks 42/142 can be integrally formed (e.g., stamping, molding, etc.) whereby they are made of the same material. The hooks 42/142 can have a variety of “hooking” shapes such as, for example, a J-shape geometry, a mushroom-shape geometry, an arrow-shape geometry, a barbed geometry, and/or a bulbous geometry. Although not specifically shown in the drawings, an adhesive layer can be provided on the side of the substrate 41/141 opposite its hook-carrying side.

The mask 80/180 and/or the fingerlift 90/190 can be made of any suitable material. For example, they can be made of cloth, kraft paper, cellophane film, non-woven webs, polymeric films (e.g., polypropylene, polyvinyl chloride, polyethylene terephthalate, and polyethylene) and/or combinations thereof. In most instances, the adhesive 32/132 will be sufficient for bonding purposes as they are usually not subjected to any forces that would encourage detachment or flagging.

One may now appreciate the fastener tab 10 is provided with an antipeel-tape-segment 60, which is formed from the same tape 170 as the release-tape-segment 50. Although the fastener tab, the tape-segments, the tapes, the laminates, and corresponding systems and methods have been shown and described with respect to a certain embodiment or embodiments, it is obvious that equivalent alterations and modifications will occur to others skilled in the art upon the reading and understanding of this specification and the annexed drawings. In regard to the various functions performed by the above described elements (e.g., components, assemblies, systems, devices, compositions, etc.), the terms (including a reference to a “means”) used to describe such elements are intended to correspond, unless otherwise indicated, to any element that performs the specified function of the described element (i.e., that is functionally equivalent), even though not structurally equivalent to the disclosed structure that performs the function. In addition, while a particular feature of the invention may have been described above with respect to only one or more of several illustrated embodiments, such a feature may be combined with one or more other features of the other embodiments, as may be desired and advantageous for any given or particular application.

Accordingly, the foregoing detailed description of the present invention is provided for purposes of illustration, and it is not intended to be exhaustive or to limit the invention to the particular embodiments disclosed. The embodiments may provide different capabilities and benefits, depending upon the configuration used to implement the key features of the invention. Accordingly, the scope of the invention is defined only by the following claims.

1. A fastener tab in a pre-deployment condition that is configured to be coupled to a diaper having a diaper edge with a first surface and a second surface, the fastener tab comprising:
a. a fastening-tape-segment including:
i. a first anchoring zone that is configured to anchor the fastener tab to the first surface of the diaper edge, and
ii. a deployment zone that is configured to be pulled away from the second surface of the diaper edge and to deploy the fastener tab after a deployment force is applied to the fastener tab;
b. a hook-tape-segment that is attached to the deployment zone;
c. a release-tape-segment including:
i. a second anchoring zone having a leading edge, the second anchoring zone is configured to anchor the fastener tab to the second surface of the diaper edge, and
ii. a hinge zone that is attached to the deployment zone; and
d. an antipeel-tape-segment that is attached to the deployment zone;
e. wherein:
i. the antipeel-tape-segment is positioned to prevent the leading edge of the second anchoring zone from being subjected to the deployment force, and
ii. the release-tape-segment and the antipeel-tape-segment are formed from a single tape.

2. The fastener tab according to claim 1, wherein:
a. the fastening-tape-segment has a first side;
b. the hinge zone and the antipeel-tape-segment overlie the first side of the fastening-tape-segment; and
c. the second anchoring zone overlies the hinge zone and the antipeel-tape-segment.

3. The fastener tab according to claim 1, further comprising at least one mask-segment that is attached to the deployment zone.

4. The fastener tab according to claim 1, wherein the fastening-tape-segment has a distal edge, and further comprising a fingerlift that is attached to the deployment zone of the fastening-tape-segment adjacent its distal edge.

5. The fastener tab according to claim 1, further comprising an antiflag adhesive area that is configured to releasably adhere the deployment zone to the release-tape-segment or the second surface of the diaper edge.

6. The fastener tab according to claim 1, wherein:
a. the fastening-tape-segment includes a substrate and an adhesive layer that is coupled to the substrate; and
b. the hook-tape-segment, the release-tape-segment, and the antipeel-tape-segment are attached to the adhesive layer.

7. The fastener tab according to claim 5, wherein the antiflag adhesive area is configured to releasably adhere the deployment zone and the second anchoring zone together.

8. A method of making a fastener tab, the method comprising:
a. providing a single tape having a cross direction;
b. partitioning the single tape in the cross direction into a release tape and an antipeel tape;
c. compiling a tape laminate including a fastening tape, a hook tape, the release tape, and the antipeel tape, the tape laminate having a machine direction; and
d. segmenting the tape laminate in the machine direction into the fastener tab.

9. The method according to claim 8, wherein the tape-compiling step includes attaching the hook tape to the fastening tape.

10. The method according to claim 8, wherein the tape-compiling step includes attaching the single tape to the fastening tape before the single-tape-partitioning step.

11. The method according to claim 8, wherein the tape-compiling step includes compiling a masking strip that is segmented into a mask-segment during the laminate-segmenting step.

12. The method according to claim 8, wherein the tape-compiling step includes compiling a fingerlift strip that is segmented into a fingerlift during the laminate-segmenting step.

13. A method according to claim 8, wherein:
a. the single tape includes an intermediate zone, an end zone, and an opposite end zone;
b. the single tape partitioning step includes partitioning the opposite end zone from the intermediate zone and the end zone; and
c. the intermediate zone and the end zone form the release tape, and the opposite end zone forms the antipeel tape.

14. The method according to claim 13, wherein the single tape has a designed weakness between the intermediate zone and the opposite end zone.

15. The method according to claim 14, wherein the single-tape-partitioning step includes breaking the designed weakness.

16. The method according to claim 13, wherein the single tape includes a fold line between the intermediate region and the end zone, and further comprising the step of folding the single tape at the fold line.

17. The method according to claim 8, further comprising the step of winding the compiled tape laminate into a roll.

18. The method according to claim 17, wherein the roll is compatible with automatic dispensing and diaper installation equipment.

19. A diaper comprising:
a. a backsheet;
b. a topsheet that is coupled to the backsheet forming a diaper edge having a first surface and a second surface;
c. a liquid absorbent core enclosed within a combination formed from the topsheet and the backsheet; and
d. at least one fastener tab including:
i. a fastening-tape-segment including:
A. a first anchoring zone that is configured to anchor the fastener tab to the first surface of the diaper edge, and
B. a deployment zone that is configured to be pulled away from the second surface of the diaper edge and to deploy the fastener tab after a deployment force is applied to the fastener tab,
ii. a hook-tape-segment that is attached to the deployment zone,
iii. a release-tape-segment including:
A. a second anchoring zone having a leading edge, the second anchoring zone is configured to anchor the fastener tab to the second surface of the diaper edge, and
B. a hinge zone that is attached to the deployment zone, and
iv. an antipeel-tape-segment that is attached to the deployment zone,
v. wherein:
A. the antipeel-tape-segment is positioned to prevent the leading edge of the second anchoring zone from being subjected to the deployment force, and
B. the release-tape-segment and the antipeel-tape-segment are formed from a single tape.

20. The method according to claim 14, wherein the single tape includes a fold line between the intermediate region and the end zone, and further comprising the step of folding the single tape at the fold line.
